(19) United States
(12) Patent Application Publication
(10) Pub. No.: US 2008/0319087 A1
(43) Pub. Date: Dec. 25, 2008

(54) Use of Ambroxol for the Treatment of Rhinovirus Infections

(76) Inventors: Anke Esperester, Mainz (DE); Liane Paul, Muenchen (DE)

Correspondence Address: MICHAEL P. MORRIS;BOEHRINGER INGELHEIM USA CORPORATION, 900 RIDGEBURY RD, P. O. BOX 368, RIDGEFIELD, CT, 06877-0368, US

(21) Appl. No.: 12/063,874

(22) PCT Filed: Aug. 3, 2006
(86) PCT No.: PCT/EP2006/064999

§ 371 Date: Jul. 29, 2008

(30) Foreign Application Priority Data
Aug. 25, 2005 (EP) 05107792.3

(51) Int. Cl. A61K 31/138

(52) U.S. Cl. 514/655

(57) ABSTRACT

The invention is directed to the use of ambroxol or pharmaceutically acceptable salts thereof for preparing a medical composition for treating or preventing infections caused by human rhinovirus. Surprisingly it was found that Ambroxol is capable to suppress the replication of human rhinovirus. Therefore, the ambroxol-containing medical composition is suitable to treat or prevent against all symptoms of a rhinovirus infection and provides a direct antivirale effect.

### FIELD OF THE INVENTION

The present invention is directed to the novel use of Ambroxol (trans-4-(2-amino-3,5-dibromobenzylamino)-cyclohexanole) or pharmaceutically acceptable salts thereof in medical compositions.

### BACKGROUND OF THE INVENTION

Human rhinovirus accounts for 30-50% of colds per year. Although rhinovirus infection is usually benign in nature it represents a significant economic burden on society in terms of absences from work and costs through visits to doctors, medicaments and other treatments (Heikkinen T, Jarvinen A, THE LANCET 361, 51-59 (2003)).

Various medicaments are used to alleviate the symptoms of the common cold. Decongestants, for instance, reduce nasal blockage and discharge. Local anaesthetics ease the pain in sore throat and cough remedies may suppress tickling or support expectoration of phlegm. Until now, there is no treatment available in the market, that is directed against the rhinovirus itself, as the cause of most upper respiratory infections.

It is known that the human rhinovirus belongs to the family of picornaviruses. Picornaviruses are characterised by a genome consisting of RNA, packed into a protein capsid.

Rhinoviruses affect the nasopharyngeal area, where they get access to the epithelial cells using intercellular adhesion-molecule-1 (ICAM-1) as their receptor. Following adherence to the receptor and subsequent uncoating the virus penetrates into the cell and starts to replicate. There are a few antiviral agents under development that aim to interfere with rhinovirus replication. The first agent for which a new drug application was filed in 2001 is pleconaril, a compound that inhibits attachment and/or virus uncoating. (Arruda et al., Antimicrob. Agents Chemother. 361186-1191 (1992)). Up to now, pleconaril has not received marketing authorisation.

A beneficial effect of ambroxol in certain viral infections is described in the prior art. Ambroxol was shown to increase the survival rate of mice infected with influenza A virus by stimulating the body's own defensive mechanisms (Yang et al., Eur. Resp. J. 19, 952-958 (2002). However, a direct effect of ambroxol on virus replication has not been shown so far. In addition, influenza A virus belongs to a class of viruses having a different structure and accordingly different ways of infecting cells compared to picornaviruses.

Furthermore EP 0 240 907 describes the use of ambroxol or a pharmaceutically suitable salt thereof for the preparation of a medicament which decongests the nasal mucosa for the local treatment of rhinitis, in the form of a nasal spray or nasal drops. However, “Rhinitis” is a term describing the symptoms produced by all kind of nasal irritation or inflammation. Symptoms of rhinitis include runny nose, itching, sneezing and stuffy nose due to blockage or congestion. These symptoms are the nose's natural response to inflammation and irritation, and they are often associated with itching of the eyes. In allergic rhinitis airborne irritants (allergens) trigger the release of histamine. Histamine causes inflammation and fluid production in the fragile linings of nasal passages, sinuses, and eyelids. The other category is nonallergic rhinitis such as vasomotor rhinitis (irritant rhinitis), rhinitis medicamentosa, neutrophilic rhinosinusitis, structural rhinitis, nasal polyps, primary vasomotor instability and the like. Causes of nonallergic rhinitis include: fumes, odors, temperature, atmospheric changes, smoke or other irritants and the reactions from nonallergic rhinitis include: sneezing, congestion, runny nose and itchy nose, throat, eyes, and ears.

However, said prior art provides a medicament which is effective against the after-effects of such a disease and may be only used in the limited local decongestant treatment of the nasal mucosa. It is not intended to be effective against the cause of such a disease which may be the rhinovirus per se which is not mentioned in EP 0 240 907.

It is therefore the object of the present invention to provide an active agent for preparing a medicament capable of the prevention or treatment of infections caused by human rhinovirus. Furthermore preparations or formulations of said compound should be provided, that are specifically suitable to be used as a treatment or prophylaxis for rhinovirus infections.

### DESCRIPTION OF THE INVENTION

The present invention concerns the use of ambroxol or pharmaceutically acceptable salts thereof for preparing a medical composition for treating or preventing infections caused by human rhinovirus (HRV), such as common cold. The invention further relates to preparations made of said compound, that are specifically suitable to be used as a treatment or prophylaxis for rhinovirus infections.

Ambroxol is a colourless and odourless substance having a slightly bitter taste. The local compatibility thereof has found to be very good and the prepared formulations have a superior effectivity without adverse side effects. Therefore the medical compositions are suitable to be administered also to aged patients, infants or babys. The formulations according to the present invention contain Ambroxol having the chemical formula

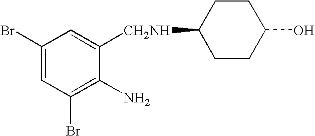

or preferable the watersoluble salts thereof may be used.

However, although ambroxol is known to be effective against infections with influenza A virus or other viruses containing a glycoprotein membrane it should be noted that the rhinovirus belongs to a totally different group of viruses which have only one protein capsid. Human rhinovirus belongs to the family of picornaviruses. An effectivity against one group of viruses does not automatically lead to the conclusion that an agent may be effective for any other virus infection. Actually, influenza A virus belongs to a class of viruses having a different structure and accordingly different ways of infecting cells compared to picornaviruses. Therefore, the mechanism and effectivity of ambroxol against human rhinovirus was completely unexpected.

Surprisingly it was found that Ambroxol is capable to suppress the replication of human rhinovirus.

It is totally unexpected that Ambroxol has a direct anti-viral influence being directly effective against the rhinovirus. In prior art EP 0 240 907, mentioned above, the disease has already manifested and the noticeable symptoms dramatically increase by-and-by, so that the treatment with a medicament becomes necessary. In contrast, the medicament of the present invention may be administered as the disease approaches or as prevention if no symptoms are noticed. Therefore, the use of the formulation according to the present invention is not intended for a medicament which decongests the nasal mucosa for the local treatment of rhinitis, in the form of a nasal spray or nasal drops. The present invention is intended to be effective against the cause of said disease, particularly the rhinovirus as it is, which is not related to limited local treatment of the nasal mucosa but it is a systemic treatment of the symptoms, if present, of the entire human body.

Ambroxol or the pharmaceutically acceptable salt thereof is used to prepare a medical composition which can be administered to a patient in a variety of dosage forms, like the usual pharmaceutical compositions. The local administration on the infected sites depends from the symptoms of the patient. For instance, the medical composition may be prepared for oral and/or nasal application.

Preferred formulations would be solid, semisolid, liquid or another dosage form. It is clear for the skilled person that the formulation may be prepared using state-of-the-art excipients and applying usual pharmaceutical technologies.

The dosage form may be a solid medical composition such as tablets or coated tablets, powders, fine granules, granules, capsules e.g. hard or soft gelatin capsules, troches (pastilles), a bolus and chewable preparations containing ambroxol or a pharmaceutically acceptable salt thereof.

Further preferred formulations may be a semisolid or liquid dosage form such as gel, e.g. a hydrogel, a cream, an ointment, a lotion, water-in-oil or oil-in-water emulsions, suspensions, aerosols, and liquid preparations such as solutions, elixirs, syrups including dry syrups. Most preferred is a liquid preparation for inhalation or a liquid preparation for rinsing of the nasal cavity.

Another dosage form suitable according to the present invention is a liquid spray or a nebulized powder allowing to bring the active ingredient into the nasal or nasopharyngeal cavity. Most preferred would be a liquid in form of a nasal spray.

In case where it is desired to ensure more rapid and reliable efficacy through the selection of local administration, the ambroxol being contained in a medical composition used according to the present invention may be administered according to the methods conventionally used such as injection of liquid preparations, spraying of mist, injection using a nebulizer, the administration by a dry powder device (DPD) using a spinhaler or a diskhaler or the administration by a metered dose inhaler (MDI). In this respect, these methods are selected and used while taking into consideration, for instance, facilities, reliability and effectiveness.

Regarding the powdery and granular preparations such as the powders, fine granules and granules, including those administered by a metered dose inhaler (MDI) or a dry powder device (DPD), they may appropriately be prepared, while taking into consideration various properties such as the dustability and adhesiveness. For instance, they are preferably prepared, while taking into consideration physical properties thereof such as the bulk, dustability, adhesiveness, hygroscopicity, charging ability, wettability and solubility of each powdery substance as well as other properties such as the particle size (particle diameter), surface area, and shapes of particles.

Specifically, in the powder inhalation, one should pay special attention to the particle size of the drug components in order to effectively make the drug arrive at the affected site and accordingly, the most suitable particle size thereof should range from 0.5 to 5.0 μm. Moreover, it is also preferred to prepare the composition while taking into consideration, for instance, the easy handling ability, and prevention of hygroscopicity, decomposition behaviors, denaturation and discoloration. The powder may be prepared according to any known pulverization method such as dry pulverization, wet pulverization, low temperature pulverization, jet pulverization, batchwise pulverization, continuous open circuit-pulverization and continuous closed circuit-pulverization methods, which may be used alone or in any combination, depending on purposes.

The preparation of pharmaceutical forms of the above-mentioned kind is well-known per se from the prior art.

The dose or dosage of the ambroxol containing medical composition of the invention to be administered may appropriately be controlled depending on the dosage forms of the desired pharmaceutical preparations.

The ambroxol containing medical composition of the invention may be administered to a patient in a daily dose in portions over one or several times per day if it is in the dosage form of an orally administered solid preparation such as a tablet or an orally or nasally administered liquid preparation. In case of the dosage forms, for infants, to be taken at one dose, such as a syrup, a troche and a chewable tablet, which are pharmaceutical preparations for simultaneously enjoying their local effects and systemic effects through the internal use thereof, it can be sufficient to incorporate ½ to 1/10 time of the daily dose of the agent in the foregoing dosage forms. In this case, the total dose thereof may be less than the daily dose.

The amount of the effective substance may also be formulated into a single dose, in as much as it is not unreasonable from the viewpoint of the dosage form of the pharmaceutical preparation. In case of, for instance, an injectable liquid preparation, a mist-spray device, a nebulizer or a powder inhalation is used, the active substance may be prepared in such a manner that the effective substance is administered in an amount of 1/10 to 1/100 time the dose for the orally administered agent for internal use.

Therefore, a solid dosage form such as a capsule, tablet, pastille, granule, a powder or a liquid or another dosage form for oral application preferably contains ambroxol or a pharmaceutically acceptable salt thereof in amounts allowing to provide 15 to 250 mg, preferably 0.30 to 150 mg, particularly 60 to 120 mg of the active ingredient per single dose.

A semisolid or liquid dosage form such as a gel, a cream or an ointment or a liquid spray or a nebulized powder preferably contains 0.1 to 10%, more preferably 0.5 to 5%, most preferably 0.5 to 3%, particularly 1 to 3% of ambroxol or a pharmaceutically acceptable salt thereof allowing to bring the active ingredient into the nasal or nasopharyngeal cavity.

A liquid dosage form for inhalation or a liquid for rinsing of the nasal cavity preferably contains 0.1 to 10%, more preferably 0.5 to 5%, most preferably 0.75 to 3%, particularly 0.75% to 1% of ambroxol or a pharmaceutically acceptable salt thereof.

Another liquid dosage form, for example, a nasal spray, preferably contains 0.1% to 10%, more preferably 0.5 to 5%, most preferably 0.5 to 2%, particularly 0.5 to 1% of ambroxol or a pharmaceutically acceptable salt thereof. Ideally the nasal spray is administered using a metered dose pump delivering between 100 and 200 μl of liquid.

In the preparation of the ambroxol containing composition, a variety of currently used additives may be employed, such as one or more of a filler, a thickening agent, a gelling agent, a binder, a disintegrator, a surfactant, a lubricant, a coating agent, a sustained release agent, a diluent and/or one or more excipients. In addition to the foregoing, the agent of the present invention may, if necessary, further comprise other additives such as a solubilizing agent, a buffering agent, a preservative, an isotonic agent, an emulsifying agent, a suspending agent, a dispersant, a hardening agent, an absorbent, an adhesive, an elasticizing agent, an adsorbent, a perfume, a coloring agent, a corrigent, an antioxidant, a humectant, a light-screening agent, a brightener, a viscosity enhancer, an oil, a tabletting adjuvant, and/or an anti-static agent.

More specifically, examples of such additives include one or more excipients such as lactose, corn starch, mannitol, D-sorbitol, crystalline cellulose, erythritol and sucrose; a binder such as hydroxypropyl cellulose (HPC-L), hydroxypropyl methyl cellulose, polyvinyl pyrrolidone, methyl cellulose and gelatinized starch; a disintegrator such as calcium carboxymethyl cellulose, crosslinked sodium carboxymethyl cellulose and crosslinked polyvinyl pyrrolidone (crospovidon); a lubricant such as magnesium stearate and talc; a perfume, for instance, a flavor or an aromatic oil such as apple essence, honey flavour, l-menthol, vanillin, lemon oil, cinnamon oil, mentha oil or peppermint oil; and/or an adsorbent such as synthetic aluminum silicate and light anhydrous silicic acid.

Moreover, it is also possible to prepare coated pharmaceutical preparations through the use of a currently used coating agent such as hydroxypropyl methyl cellulose, hydroxypropyl cellulose, methyl cellulose or polyvinyl pyrrolidone.

If necessary, a sweetener may likewise be used, in particular, in troches, syrups and chewable preparations among others. Specific examples of such sweeteners are mannitol, glucose, maltose, starch syrup, malt extract, maltitol, sorbitol, sucrose, unrefined sugar, fructose, lactose, honey, xylitol, hydrangea tea, saccharin, aspartame, cyclamate, Sunett®, aspartyl phenylalanine ester and other malto-oligo saccharides, and oligo saccharides such as maltosyl sucrose, isomaltyrose of reduced type and raffinose, Acesulfame potassium or any kind of sugar alcohols or mixtures thereof such as sorbitol, mannitol and/or xylitol.

As solubilisers any known solubiliser suitable in the medical sector may be used, for example polyethyleneglycols, polyoxyethylene-polyoxypropylene copolymers (e.g. poloxamer 188), glycofurol, arginine, lysine, castor oil, propyleneglycol, solketal, polysorbate, glycerol, polyvinyl pyrrolidone, lecithin, cholesterol, 12-hydroxystearic acid-PEG660-ester, propyleneglycol monostearate, polyoxy-40-hydrogenated castor oil, polyoxyl-10-oleyl-ether, polyoxyl-20-ceto-stearylether and polyoxyl-40-stearate or a mixture thereof.

Any preservatives known for use in the pharmaceutical field may be used, for example, ethanol, benzoic acid and the sodium or potassium salts thereof, sorbic acid and the sodium or potassium salts thereof, chlorobutanol, benzyl alcohol, phenylethanol, methyl-, ethyl-, propyl- or butyl-p-hydroxybenzoates, phenol, m-cresol, p-chloro-m-cresol, those selected from the group of the PHB esters, e.g. mixtures of PHB-methyl with PHB-propylesters, quaternary ammonium compounds such as benzalkonium chloride, thiomersal, phenyl-mercury salts such as nitrates, borates.

The buffer system used to achieve a desired pH value may be, for example, glycine, a mixture of glycine and HCl, a mixture of glycine and sodium hydroxide solution, and the sodium and potassium salts thereof, a mixture of potassium hydrogen phthalate and hydrochloric acid, a mixture of potassium hydrogen phthalate and sodium hydroxide solution or a mixture of glutamic acid and glutamate.

Suitable gelling agents are for example cellulose and its derivatives, like for instance methyl cellulose, carboxymethyl cellulose, hydroxypropylmethyl cellulose, poly(vinyl)alcohol, poly(vinyl)pyrrolidones, polyacrylates, poloxamers, tragacanth, carrageenan, starch and its derivatives or any other gelling agent used in pharmaceutical technology.

Viscosity enhancers which may be mentioned are for example the aforementioned gelling agents in low quantities, glycerol, propylene glycole, polyethylene glycol or polyols, like sorbitol and other sugar alcohols.

The preferred emulsifiers used, apart from the emulsifiers known from the prior art, include polyoxyethylene derivatives of castor oil or polyoxyethylene alkylethers.

Suitable synthetic or natural, colouring agents known in the pharmaceutical field may be used such as Indigo carmine.

Suitable oily components which may be present are any of the oily substance known from the prior art for the preparation of pharmaceuticals, such as, for example, vegetable oils, in particular, e.g. cotton seed oil, groundnut oil, peanut oil, maize oil, rapeseed oil, sesame oil and soya oil, or triglycerides of moderate chain length, e.g. fractionated coconut oil, or isopropylmyristate, -palmitate or mineral oils or ethyloleate.

The antioxidants used may be any of the antioxidants known from the prior art, preferably a-tocopherol, butylhydroxytoluene (BHT) or butylhydroxyanisole (BHA).

Pharmaceutical preparations containing these additives may be prepared according to any method known in this field, currently used ones or ordinary ones depending on the dosage forms thereof. It is a matter of course that further additives not explicitely discussed may be used in the formulations used according to the present invention.

Although the preparation of pharmaceutical forms of the above-mentioned kind is well-known per se from the prior art, particularly preferred exemplary ambroxol containing medical compositions will be described in the following.

An exemplary tablet formulation may for instance be prepared using as excipient cellulose or sweeter(s) such as sugar or sugar alcohols or as tabletising adjuvant sodium phosphates as a tablet base. Other excipients may be polyethylene glycols and disintegrants, such as cross-linked polyvinyl pyrrolidone and lubricants, like stearic acid, fumaric acid and salts thereof.

An exemplary pastille may be prepared using acacia gum, modified starch, carrageen or gelatine, i.e. binder(s), thickener(s) and gelatinizing additive(s) as the base.

A preferred embodiment according to the present invention is a capsule such as a hard gelatine capsule size 1 containing or essentially consisting of:

30 to 75 mg Ambroxol hydrochloride;
150 to 300 mg filler excipients;
2 to 15 mg lubricant(s).

Another preferred embodiment according to the present invention are granules to be dissolved in a liquid for oral application containing or essentially consisting of:

30 to 60 mg Ambroxol hydrochloride;
1000 to 3000 mg excipient(s);
5 to 20 mg sweetener(s) and
optionally 20 to 60 mg flavour(s); and

Another preferred embodiment according to the present invention is a gel to be applied to the nasal cavity containing or essentially consisting of:

50 to 200 mg Ambroxol hydrochloride
50 to 1000 mg gelling agent;
optionally 1000 to 3000 mg polyol;
optionally 3 to 20 mg flavour(s);
optionally 10 to 50 mg preservative(s); and
5730 to 8887 mg water.

Another preferred embodiment according to the present invention is a solution for inhalation containing or essentially consisting of:

100 to 200 mg Ambroxol hydrochloride;
20 to 80 mg buffer substances;
40 to 60 mg isotonic agent(s);
and
9650 to 0.9800 mg water.

The term “solution” should be understood in the frame of the present invention to comprise any dispersed system, true solutions as well as any intermediate states.

Another preferred embodiment according to the present invention is a solution for a nasal spray containing or essentially consisting of:

50 to 200 mg Ambroxol hydrochloride;
250 to 500 mg sorbitol;
20 to 40 mg monosodium phosphate;
3 to 7 mg disodium hydrogene phosphate;
15 to 20 mg benzalkonium chloride
and
9600 to 9700 mg water.

Some formulations by way of example follow the experimental section.

The advantages of the present invention are manifold:

It is provided the use of ambroxol or pharmaceutically acceptable salts thereof for preparing a medical composition for treating or preventing infections caused by human rhinovirus such as common cold. It has surprisingly found that ambroxol is capable to suppress the replication of human rhinovirus.

The ambroxol containing medical composition may be administered independent from the selected dosage form which is variable according to the disease and/or symptoms to be treated. Diseases such as common cold caused by rhinovirus infections are harmless but associated with inconvenient concomitant symptoms. The severity of symptoms may be dramatically alleviated by the treatment with a suitable ambroxol containing medicament and the duration of the disease will be extremely shortened.

Ambroxol was found to be effective against rhinovirus in vitro and the described medical compositions result in concentrations at the affected sites which correspond to the concentrations found in the in vitro experiments.

Ambroxol has an outstanding compatibility, it shows practically no side-effects and the dosage forms are suitable for selfmedication.

Therefore, the ambroxol-containing medical composition is suitable to treat or prevent against all symptoms of a rhinovirus infection and provides a direct antivirale effect.

### EXAMPLES

### Example 1

### Capsules to be Swallowed

TABLE 1

| igredient | amount | |
|---|---|---|
| Ambroxol hydrochloride | 75 | mg |
| Corn starch | 100 | mg |
| Lactose monohydrate | 180 | mg |
| Magnesium stearate | 2 | mg |

Filled into hard gelatine capsules size 1.

### Example 2

### Granules to be Dissolved in Liquid for Oral Application

TABLE 2

| ingredient | amount | |
|---|---|---|
| Ambroxol hydrochloride | 60 | mg |
| Sorbitol | 2750 | mg |
| Acesulfame potassium | 15 | mg |
| Vanilla aroma | 20 | mg |
| Strawberry aroma | 30 | mg |

### Example 3

### Gel to be Applied to the Nasal Cavity

TABLE 3

| ingredient | amount | |
|---|---|---|
| Ambroxol hydrochloride | 0.4 | g |
| Hydroxyethyl cellulose | 1.0 | g |
| Citric acid | 0.02 | g |
| Glycerol 85% | 4.0 | g |
| Peppermint oil | 0.005 | g |
| Benzalkonium chloride | 0.05 | g |
| Water | 14.525 | g |

### Example 4

### Solution for Inhalation

TABLE 4

| ingredient | amount | |
|---|---|---|
| Ambroxol hydrochloride | 1.0 | g |
| Citric acid monohydrate | 0.2 | g |
| Sodium hydroxide | 0.07 | g |
| Sorbitol | 10.0 | g |
| Water | 88.73 | g |

### Example 5

### Solution for a Nasal Spray

TABLE 5

| ingredient | amount | |
|---|---|---|
| Ambroxol hydrochloride | 0.75 | g |
| Citric acid monohydrate | 0.01 | g |
| Disodium phosphate | 0.018 | g |
| Sodium chloride | 0.07 | g |
| Water | 9.152 | g |

### Example 6

### Description of Antiviral Testing and Test Results

For the purpose of the study HeLa cells were inocculated with human rhinovirus subtype 14 (HRV 14), isolated from nasopharyngeal secretions. Ambroxol hydrochloride (AX) was chosen as the compound for antiviral testing. The antiviral activity of the test substance was evaluated in the plaque-reduction assay.

As a first step an assay on possible toxic effects of AX was performed by incubating HeLa cells with increasing dilutions of the test substance for 5 days.

Cell morphology was checked using a microscope. In addition, an assay for mitochondrial enzyme activity (MTT-assay) was used to evaluate the vitality of cells. No changes in cell morphology and enzyme activity were found at concentrations of 10 μg AX/ml and below.

To test the effect of AX on rhinovirus replication HeLa cells were infected with a multiplicity of infection (MOI) of 0.0002, without (control for 100% infection) or in the presence of AX at concentrations between 0.3 and 10 μg/ml. Infected cell cultures were cultivated for three days. Inhibition of virus plaques was evaluated microscopically and compared with controls.

The results were as follows:

TABLE 6

| Ambroxol hydrochloride | relative inhibition |
|---|---|
| Concentration μg/ml | %* |
| 10 | 70.65 |
| 5 | 63.66 |
| 2.5 | 36.75 |
| 1.2 | 34.31 |
| 0.6 | 14.45 |
| 0.3 | 9.71 |

*average of 6 individual tests

From these results it can be concluded that AX effectively blocks rhinovirus replication.

Rhinovirus replication in vivo takes predominantly place in the nasopharynx. In order to be effective as an antiviral agent AX would have to be available at the site of infection in a sufficiently high concentration. The invention therefore also relates to preparations that would allow to provide AX at the target area.

1.-9. (canceled)

10. A method for preventing rhinovirus infection in a human which comprises administering a prophylacticn amount of ambroxol or a pharmaceutically acceptable salt thereof to said human.

11. The method as recited in claim 10 wherein the ambroxol or ambroxol salt is administered orally or nasally.

12. The method as recited in claim 10 wherein the amount of ambroxol or ambroxol salt administered is between about 15 mg to about 250 mg.

13. A method for treating rhinovirus infection in a human which comprises administering a therapeutically effective amount of ambroxol or a pharmaceutically acceptable salt thereof to said human.

14. The method as recited in claim 13 wherein the ambroxol or ambroxol salt is administered orally or nasally.

15. The method as recited in claim 13 wherein the amount of ambroxol salt administered is between about 15 mg to about 250 mg.
